(19) United States
(12) Patent Application Publication
(10) Pub. No.: US 2019/0224332 A1
(43) Pub. Date: Jul. 25, 2019

(54) STERILE INJECTABLE COMPOSITIONS COMPRISING DRUG MICELLES

(71) Applicant: CADILA HEALTHCARE LIMITED, Ahmedabad, Gujarat (IN)

(72) Inventors: Sushilkumar Dhanaji PATIL, Ahmedabad, Gujarat (IN); Smita Amarjitsing RAJPUT, Ahmedabad, Gujarat (IN); Nirav Ishwarlal KHATRI, Ahmedabad, Gujarat (IN); Alex Kochukunju GEORGE, Ahmedabad, Gujarat (IN); Sushrut Krishnaji KULKARNI, Ahmedabad, Gujarat (IN)

(73) Assignee: CADILA HEALTHCARE LIMITED, Ahmedabad, Gujarat (IN)

(21) Appl. No.: 16/330,478

(22) PCT Filed: Sep. 6, 2017
(86) PCT No.: PCT/IB2017/055365

§ 371 Date: Mar. 5, 2019

(30) Foreign Application Priority Data
Sep. 7, 2016 (IN) 201621030489
Jun. 27, 2017 (IN) 201721022389

(51) Int. Cl. A61K 47/69; A61K 9/107; A61K 9/00; A61K 31/337; A61K 9/19; A61K 47/32; A61K 47/24

(52) U.S. Cl. CPC A61K 47/6909; A61K 9/1075; A61K 9/0019; A61K 47/24; A61K 9/19; A61K 47/32; A61K 31/337

(57) ABSTRACT

The present invention relates to sterile injectable compositions comprising drug-phospholipid micelles. The present invention also relates to processes for the preparation of the sterile injectable compositions.

### FIELD OF THE INVENTION

The field of the present invention relates to sterile injectable compositions comprising drug-phospholipid micelles. The present invention also relates to processes for the preparation of the sterile injectable compositions.

### BACKGROUND OF THE INVENTION

Cancer is a group of diseases involving abnormal cell growth with the potential to invade or spread to other parts of the body. Chemotherapy is a category of cancer treatment that uses at least one anti-cancer drag (chemotherapeutic agent) as part of a standardized chemotherapy regimen.

U.S. Pre-grant Publication No. 2004/0013717 A1 discloses PEG-lipid containing formulations to deliver a photosensitizer as a pharmaceutical, agricultural, or industrial agent. It discloses formulations comprising verteporfin and PEG₂₀₀₀-DSPE conjugate as single phospholipid ingredient, prepared using organic solvents like dichloromethane.

Jing et al. (Asian Journal of Pharmaceutical Sciences; Volume 10, Issue 2, April 2015, Pages 81-98) relates to review on phospholipids and their main applications in drug delivery systems. Amongst multiple applications it discloses the use of phospholipid for preparation of micelles which involve the addition of egg phosphatidylcholine into a PE-PEG micellar system.

Helmut et al. (Colloids and Surfaces A: Physicochemical and Engineering Aspects; Volumes 183-185, 15 Jul. 2001, Pages 495-504) relates to bilayer fragments and bilayered micelles (bicelles) of dimyristoylphosphatidylglycerol (DMPG) inducement by storage in distilled water at 4° C.

Docetaxel is an anti-cancer drug, belongs to a class called plant alkaloids. Docetaxel is a clinically well-established anti-mitotic chemotherapy medication used mainly for the treatment of breast, ovarian and non-small cell lung cancer. Since docetaxel exhibits very low water solubility, its marketed product (Taxotere®, Sanofi Aventis, Dagenham, UK) for intravenous administration contains a high concentration of polysorbate 80. Yadav et al. (AAPS PharmSciTech, Vol. 16, No. 4, August 2015; 855-864) reports that, the presence of polysorbate 80 in Taxotere® causes numerous adverse effects, including acute hypersensitivity reactions, fluid retention, and peripheral neuropathy. Yadav et al. also reports that several approaches have been investigated to eliminate the addition of the polysorbate 80 and increase docetaxel solubility and efficacy. Chinese Patent Application No. CN101439033A discloses docetaxel lipid complexes prepared from docetaxel, phospholipids and cholesterol sulfate and/or cholesterol derivatives.

Yadav et al. (AAPS PharmSciTech, Vol. 16, No. 4, August 2015; 855-864) discloses docetaxel phospholipid nanoparticles.

Yousefi et al. (Sci Pharm. 2009; 77:453-464) discloses nano-liposomal formulations comprising docetaxel, phospholipids and cholesterol, prepared using organic solvents such as methanol and chloroform.

Liu et al. (American Pharmacists Association J Pharm Sci 97:3274-3290, 2008) discloses colloidal formulations comprising nano-sized assemblies of a polyethylene glycol (PEG)-docetaxel conjugate.

Ahmad et al. (Journal of Nanomedicine & Nanotechnology 2015, 6:3 1000295) discloses nanosomal docetaxel lipid suspension.

Garrec et al. (J. Drug Del. Sci. Tech., 15 (2) 115-2005) discloses docetaxel-loaded polymeric micelles comprising docetaxel and poly(N-vinylpyrrolidone)-block-poly(D,L-lactide) (PVP-b-PDLLA).

Gao et al. (Molecular Pharmaceutics VOL. 5. NO. 6, 1044-1054) discloses nanoassemblies formulations comprising docetaxel, methoxy polyethylene glycoldistearoylphosphatidylethanolamine (mPEG₂₀₀₀-DSPE) and maleimide-derivatized PEG₂₀₀₀-DSPE (Mal-PEG-DSPE) using organic solvent like chloroform.

U.S. Pre-grant Publication No. 2016/0128940 A1 discloses nano-polymer micelle lyophilized preparations comprising docetaxel and methoxypolyethylene glycol-polylactic acid block copolymer carrier.

U.S. Pat. No. 8,927,592 discloses a method for treating a patient with prostate cancer which has progressed during or after treatment with docetaxel, comprising administering to said patient a dose of 20 to 25 mg/m² of cabazitaxel, or a hydrate or solvate thereof, in combination with a corticoid like prednisone and prednisolone.

Cabazitaxel is an anti-cancer drug, belongs to a class called plant alkaloids, Cabazitaxel is a clinically well-established microtubule inhibitor indicated in combination with prednisone for the treatment of patients with hormone-refractory metastatic prostate cancer previously treated with a docetaxel based treatment regimen. Since cabazitaxel exhibits very low water solubility, its marketed product (Jevtana® Kit, SANOFI AVENTIS US INC) for intravenous administration contains polysorbate 80.Polysorbate 80 is reported to be associated with unpredictable (acute) hypersensitivity reactions and cumulative fluid retention when administered parenterally.

International (PCT) Publication Number WO/2016/113752 discloses liquid formulation of cabazitaxel or a pharmaceutically acceptable salt thereof, at least one solubilizer and a solvent. It further discloses that solubilizers can be selected from the group comprising of benzyl alcohol, tertiary-butyl alcohol, isopropyl alcohol, acetic acid, glycols, polysorbates, polyoxyethylene glycol esters, polyoxyethylene castor oil derivatives and suitable mixtures thereof.

International (PCT) Publication Number WO/2013/024495 discloses a pharmaceutical formulation for parenteral administration, comprising cabazitaxel or a pharmaceutically acceptable salt thereof and at least one solubilizer. It further discloses that solubilizers can be selected from the group comprising of polysorbates, polyethylene glycols, propylene glycol, tetraglycol, glycerol, ethanol and a mixture thereof.

International (PCT) Publication Number WO/2013/022960 discloses a liquid sterile pharmaceutical formulation comprising: cabazitaxel, or a pharmaceutically acceptable salt thereof; a solubilizer selected from glycofurol and ethanol; tocopherol polyethylene glycol succinate (TPGS); at least one hydrotrope; optionally at least one agent having a pKa of about 3 to about 6; and optionally at least one antioxidizing agent. It also discloses the formulation which is substantially free of polysorbates and polyethoxylated castor oil.

There exists a need of an alternative, improved and stable injectable composition which enables efficacious and safe parenteral delivery of anti-cancer drugs, like docetaxel and cabazitaxel.

### SUMMARY OF THE INVENTION

In one general aspect the present invention provides a sterile injectable composition comprising drug-phospholipid micelles.

In another general aspect the sterile injectable composition comprises one or more drugs, one or more phospholipids and one or more stabilizers.

In another general aspect the present invention provides a sterile injectable composition comprising docetaxel-DMPG (1,2-dimyristoyl-sn-glycero-3-phosphorylglycerol) micelles or cabazitaxel-DMPG micelles.

In another general aspect the present invention provides a process for the preparation of the sterile injectable composition.

In another general aspect the invention provides a method of treating cancer by administering the sterile injectable composition of the present invention to the individual in need thereof.

The details of one or more embodiments of the invention are set forth in the description below. Other features, objects and advantages of the invention will be apparent from the description.

### DETAILED DESCRIPTION OF THE INVENTION

The present invention provides a sterile injectable composition comprising one or more drugs and one or more pharmaceutical acceptable excipients.

The sterile injectable composition of the present invention may be in the form of a stable lyophilized powder or a stable, clear, aqueous solution.

The sterile injectable composition of the present invention may be administered via parenteral route, for example, intravenous (I.V.), subcutaneous (S.C.) or intramuscular (I.M.).

The sterile injectable composition of the present invention may provide one or more advantages like, providing a safe composition by solubilizing the drug without usage of toxic ingredients such as polysorbate-80 or cremophor®, providing a stable, clear, aqueous solution suitable for I.V. administration compatible with routinely used i.v. sets and having improved stability, efficacy, and safety profile, Polysorbate-80 and cremophor are reported as the cause of hypersensitivity reactions and other unwanted effects.

The sterile injectable composition of the present invention does not comprise chotesterol or cholesterol derivatives (for example, sodium cholesteryl sulphate etc.), lecithin or its derivative, phosphatidylcholines and/or polysorbate-80.

In one embodiment, the stable, clear, aqueous solution comprises micelles and is lyophilizable. The stable, clear, aqueous solution of the present invention does not comprise bilayered micelles (bicelles). The sterile injectable composition of the invention is not a powder for suspension, liquid suspension or nanoparticulate dispersion.

In one embodiment, the sterile injectable composition is a stable lyophilized powder comprising one or more drugs and one or more pharmaceutically acceptable excipients.

In another embodiment, the stable lyophilized powder comprises one or more drugs, one or more phospholipids and one or more stabilizers.

Suitable drug for the composition of the invention may have low water solubility or may be water insoluble. Examples of suitable drug may include one or more of anticancer drugs such as taxane derivatives (docetaxel, paclitaxel, and cabazitaxel), temsirolimus, carmustine, bendamustin, melphalan, busulfan, carfilzomib, bortezomib, teniposide, ixabepilone etc. Examples of suitable drug may also include one or more of immunosuppressants such as sirolimus, tacrolimus, everolimus etc.

In one embodiment, the invention provides a sterile injectable composition comprising docetaxel which is therapeutically equivalent to the commercially available docetaxel formulation marketed under the trade name Taxotere®. The sterile injectable composition comprising docetaxel of the invention, may provide value of AUC (area under the curve for a plot of concentration of drug in plasma vs. time) between 1500 ng.h/mL and 7000 ng/mL, for example, 1800 ng.h/mL and 6700 ng.h/mL, for example, between 2100 ng.h/mL and 6400 ng.h/mL, between 2400 ng.h/mL and 6100 ng.h/mL, between 2700 ng.h/mL and 5800 ng.h/mL, between 3000 ng.h/mL and 5500 ng.h/mL, between 3300 ng.h/mL and 5200 ng.h/mL, between 3600 ng.h/mL and 4900 ng.h/mL or between 3900 ng.h/mL and 4600 ng.h/mL, when the sterile injectable composition comprising docetaxel is administered intravenously to a human at a dose of 75 mg docetaxel/m² in a 1-hour intravenous infusion.

In another embodiment, the invention also provides a sterile injectable composition comprising cabazitaxel which is therapeutically equivalent to the commercially available cabazitaxel formulation marketed under the trade name Jevtana® Kit. The sterile injectable composition comprising cabazitaxel of the invention, may provide value of mean Cₘₐₓ (maximum concentration of drug in plasma, achieved after administration) between 100 ng/mL and 350 ng/mL, for example, 110 ng/mL and 340 ng/mL, between 130 ng/mL and 320 ng/mL, 150 ng/mL and 300 ng/mL, between 170 ng/mL and 280 ng/mL, between 190 ng/mL and 260 ng/mL or between 210 ng/mL and 240 ng/mL, when the sterile injectable composition comprising cabazitaxel is administered intravenously to a human at a dose of 25 mg cabazitaxel/m² every three weeks. The sterile injectable composition comprising cabazitaxel of the invention, may provide value of mean AUC (area under the curve for a plot of concentration of drug in plasma vs. time) between 500 ng.h/mL and 1500 ng.h/mL, for example, between 600 ng.h/mL and 1400 ng.h/mL, between 700 ng.h/mL and 1300 ng.h/mL, between 800 ng.h/mL and 1200 ng.h/mL or between 900 ng.h/mL and 1100 ng.h/mL, when the sterile injectable composition comprising cabazitaxel is administered intravenously to a human at a dose of 25 mg cabazitaxel/m² every three weeks.

In one embodiment, the stable lyophilized powder for injection comprises docetaxel or cabazitaxel and one or more phospholipids.

In another embodiment, the stable lyophilized powder for injection comprises docetaxel or cabazitaxel and one or more micelle forming phospholipids, wherein the phospholipid is having fatty acid carbon chain length of 16 carbons or less, for example, 14 carbons or 12 carbons.

In another embodiment, the stable lyophilized powder for injection comprises docetaxel or cabazitaxel and 1, 2-dimyristoyl-sn-glycero-3-phosphorylglycerol (DMPG).

In another embodiment, the stable lyophilized powder comprises docetaxel or cabazitaxel, 1, 2-dimyristoyl-sn-glycero-3-phosphorylglycerol (DMPG) and one or more stabilizers.

In another embodiment, the stable lyophilized powder comprises docetaxel or cabazitaxel, 1, 2-dimyristoyl-sn-glycero-3-phosphorylglycerol (DMPG), N-(Carbonyl-methoxypolyethyleneglycol 2000)-1,2-distearoyl-sn-glycero-3-phosphoethanolamine (mPEG₂₀₀₀-DSPE) and polyvinylpyrrolidone. The weight ratio of DMPG to mPEG₂₀₀₀-DSPE may be between about 5:1 to about 1:1, for example 4:1, 3:1 or 2:1.

In another embodiment, there is provided a vial comprising stable lyophilized powder comprising 20 mg docetaxel, 120 mg DMPG, 40 mg N-(Carbonyl-methoxypolyethyleneglycol 2000)-1,2-distearoyl-sn-glycero-3-phosphoethanolamine (mPEG₂₀₀₀-DSPE), 40 mg polyvinylpyrrolidone K12 (PVP K12) and 800 mg of sucrose. The amount of DMPG present in a vial comprising stable lyophilized powder comprising docetaxel may be between about 80 mg and about 160 mg, for example, 90 mg, 100 mg, 110 mg, 120 mg, 130 mg, 140 mg or 150 mg. The weight ratio of docetaxel to DMPG may be between 1:2 and 1:10, for example, 1:4, 1:6 or 1:8. The amount of mPEG₂₀₀₀-DSPE present in a vial comprising stable lyophilized powder comprising docetaxel may be between about 10 mg and about 80 mg, for example, 15 mg, 20 mg, 25 mg, 30 mg, 35 mg, 40 mg, 45 mg, 50 mg, 55 mg, 60 mg, 65 mg, 70 mg or 75 mg. The weight ratio of docetaxel to mPEG₂₀₀₀-DSPE may be between 2:1 and 1:4, for example, 1:1, 1:2 or 1:3. The amount of PVP present in a vial comprising stable lyophilized powder comprising docetaxel may be between about 1 mg and about 200 mg, for example, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 100 mg, 150 mg or 200 mg. The weight ratio of docetaxel to PVP may be between 2:1 and 1:4, for example, 1:1, 1:2 or 1:3.

In another embodiment, there is provided a vial comprising stable lyophilized powder comprising 60 mg cabazitaxel, 360 mg DMPG, 120 mg N-(Carbonyl-methoxypolyethyleneglycol 2000)-1,2-distearoyl-sn-glycero-3-phosphoethanolamine (mPEG₂₀₀₀-DSPE), 120 mg polyvinylpyrrolidone K12 (PVP K12) and 2.4 gram of sucrose. The amount of DMPG present in a vial comprising stable lyophilized powder comprising cabazitaxel may be between about 300 mg to about 420 mg, for example, 310 mg, 320 mg, 330 mg, 340 mg, 350 mg, 360 mg, 370 mg, 380 mg, 390 mg, 400 mg or 410 mg. The weight ratio of cabazitaxel to DMPG may be between 1:2 and 1:10, for example, 1:4, 1:6 or 1:8. The amount of mPEG₂₀₀₀-DSPE present in a vial comprising stable lyophilized powder comprising cabazitaxel may be between about 25 mg and about 500 mg, for example, 50 mg, 75 mg, 100 mg, 110 mg, 120 mg, 130 mg, 140 mg, 175 mg, 200 mg, 250 mg, 300 mg, 350 mg, 400 mg, 450 mg or 500 mg. The weight ratio of cabazitaxel to mPEG₂₀₀₀-DSPE may be between 2:1 and 1:4, for example, 1:1,1:2 or 1:3. The amount of PVP present in a vial comprising stable lyophilized powder comprising cabazitaxel may be between about 25 mg and about 1000 mg, for example, 50 mg, 75 mg, 100 mg, 110 mg, 120 mg, 130 mg, 140 mg, 175 mg, 200 mg, 300 mg, 400 mg, 500 mg, 600 mg, 700 mg, 800 mg, 900 mg or 1000 mg. The weight ratio of cabazitaxel to PVP may be between 2:1 and 1:4, for example, 1:1, 1:2 or 1:3.

In one embodiment, the stable lyophilized powder is powder for solution. The stable lyophilized powder may be reconstituted using an appropriate quantity of vehicle (e.g. water for injection, 5% dextrose or 0.9% NaCl) to provide a stable, clear, aqueous reconstituted solution. The reconstituted solution may be diluted using an appropriate quantity of diluent (e.g. water for injection, 5% dextrose or 0.9% NaCl) to provide a stable, clear, aqueous diluted solution.

In one embodiment the sterile injectable composition is a stable, clear, aqueous solution comprising one or more drugs and one or more phospholipids. The solution of the invention does not form any precipitate after storage for relevant time period, for example, for 6 hours, for 8 hours, for 10 hours, for 12 hours, for 18 hours, for 24 hours or for 48 hours, at temperature 2-8° C. or at 25° C. The stable, clear, aqueous solution of the invention does not comprise liposome or nanoparticle or any other particulate drug delivery system.

In another embodiment, the sterile injectable composition is a stable, clear, aqueous solution comprising one or more drugs, one or more phospholipids and one or more stabilizers.

In another embodiment, the stable, clear, aqueous solution comprises drug-DMPG micelles and one or more stabilizers.

In another embodiment the stable, clear, aqueous solution comprises docetaxel or cabazitaxel and one or more phospholipids. The stable, clear, aqueous solution comprises docetaxel or cabazitaxel and 1,2-dimyristoyl-sn-glycero-3-phosphorylglycerol (DMPG). The solution may be lyophilizable. The solution may be used for parenteral administration. The solution for parenteral administration may be in the form of 100% water based solution and does not comprise any other solvent or liquid vehicle.

In another embodiment, the present invention provides a stable, clear, aqueous solution comprising docetaxel-DMPG micelles and polyvinylpyrrolidone, wherein the solution may have pH between about 5 and about 9, for example, about 6, about 7 or about 8. In another embodiment, the present invention provides a stable, clear, aqueous solution comprising cabazitaxel-DMPG micelles and polyvinylpyrrolidone. The aqueous solution may have pH between about 4 and about 9, for example, about 5, about 6, about 7 or about 8.

In another embodiment, the present invention provides a stable, clear, aqueous solution comprising drug-DMPG micelles and one or more stabilizers. The amount of DMPG present in the reconstituted solution may be between about 1 mg/mL and about 50 mg/mL, for example, 5 mg/mL, 10 mg/mL, 15 mg/mL, 20 mg/mL, 25 mg/mL, 30 mg/mL, 35 mg/mL, 40 mg/mL or 45 mg/mL. Use amount of mPEG₂₀₀₀-DSPE present in the reconstituted solution may be between about 1 mg/mL and about 50 mg/mL, for example, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL or 45 mg/mL. The amount of PVP present in the reconstituted solution may be between about 1 mg/mL and about 100 mg/mL, for example, 2 mg/mL, 3 mg/mL, 4 mg/mL, 5 mg/mL, 6 mg/mL, 7 mg/mL, 8 mg/mL, 9 mg/mL, 10 mg/mL, 25 mg/mL, 50 mg/mL, 75 mg/mL or 90 mg/mL.

In another embodiment, the present invention provides a stable, clear, aqueous solution comprising docetaxel-DMPG micelles and one or more stabilizers, wherein the docetaxel-DMPG micelles are present in an amount to provide docetaxel concentration between about 0.1 mg/mL and about 100 mg/mL, for example, between about 1 mg/mL and about 10 mg/mL, about 2.5 mg/mL or about 0.33 mg/mL. The amount of DMPG present in the diluted solution comprising docetaxel may be between about 0.1 mg/mL and about 5 mg/mL, for example, 0.5 mg/mL, 1 mg/mL, 1.5 mg/mL, 1.98 mg/mL, 2 mg/mL, 2.5 mg/mL, 3 mg/mL, 3.5 mg/mL, 4 mg/mL or 4.5 mg/mL. The amount of mPEG₂₀₀₀-DSPE present in the diluted solution comprising docetaxel may be between about 0.01 mg/mL and about 5 mg/mL, for example, 0.05 mg/mL, 0.1 mg/mL, 0.3 mg/mL, 0.4 mg/mL, 0.5 mg/mL, 0.66 mg/mL, 0.7 mg/mL, 0.8 mg/mL, 0.9 mg/mL, 1 mg/mL, 2 mg/mL, 3 mg/mL, 4 mg/mL or 4.5 mg/mL. The amount of PVP present in the diluted solution comprising docetaxel may be between about 0.01 mg/mL and about 10 mg/mL, for example, 0.05 mg/mL, 0.1 mg/mL, 0.3 mg/mL, 0.4 mg/mL, 0.5 mg/mL, 0.66 mg/mL, 0.7 mg/mL, 0.8 mg/mL, 0.9 mg/mL, 1 mg/mL, 2 mg/mL, 3 mg/mL, 4 mg/mL or 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL or 9.5 mg/mL.

In another embodiment, the present invention provides a stable, clear, aqueous solution comprising cabazitaxel-DMPG micelles and one or more stabilizers, wherein the cabazitaxel-DMPG micelles are present in an amount to provide cabazitaxel concentration between about 0.01 mg/mL and about 100 mg/mL, for example, between about 0.05 mg/mL and about 10 mg/mL, between about 0.05 mg/mL and about 5 mg/mL, about 0.1 mg/mL, about 0.26 mg/mL or about 2.5 mg/mL. The amount of DMPG present in the diluted solution comprising cabazitaxel may be between about 0.1 mg/mL and about 5 mg/mL, for example, 0.5 mg/mL, 0.75 mg/mL, 1.0 mg/mL, 1.2 mg/mL, 1.4 mg/mL, 1.5 mg/mL, 1.56 mg/mL, 1.7 mg/mL, 2 mg/mL, 3 mg/mL, 4 mg/mL or 4.5 mg/mL. The amount of mPEG₂₀₀₀-DSPE present in the diluted solution comprising cabazitaxel may be between about 0.01 mg/mL and about 5 mg/mL, for example, 0.05 mg/mL, 0.1 mg/mL, 0.3 mg/mL, 0.4 mg/mL, 0.52 mg/mL, 0.6 mg/mL, 0.7 mg/mL, 0.8 mg/mL, 0.9 mg/mL, 1 mg/mL, 2 mg/mL, 3 mg/mL, 4 mg/mL or 4.5 mg/mL. The amount of PVP present in the diluted solution comprising cabazitaxel may be between about 0.01 mg/mL and about 10 mg/mL, for example, 0.05 mg/mL, 0.1 mg/mL, 0.3 mg/mL, 0.4 mg/mL, 0.52 mg/mL, 0.6 mg/mL, 0.7 mg/mL, 0.8 mg/mL, 0.9 mg/mL, 1 mg/mL, 2 mg/mL, 3 mg/mL, 4 mg/mL or 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL or 9.5 mg/mL.

The stable, clear, aqueous solution may be in the form of micellar solution comprising docetaxel or cabazitaxel, one or more phospholipids and one or more stabilizers.

### Definitions:

As used herein, and unless otherwise specified, the term “stable” refers to the stability of the product or the composition comprising one or more drugs having sufficient physical and chemical stability for a relevant period of time under the specified storage conditions.

The term “physical stability” with respect to clear solution refers to maintenance of clear state without any drug precipitation, maintenance of color or colorless state and/or maintenance of dissolved oxygen level. The term “physical stability” with respect to lyophilized powder refers to maintenance of aesthetic appearance, absence of agglomerates, and maintenance of flowability, dispersibility and water content.

The term “chemical stability” relates to maintenance of drug-related impurities in terms of total impurities, known impurities, single maximum known impurity and single maximum unknown impurity, within the allowed limits by the regulatory agency.

The stable lyophilized powder of the invention remains stable for commercially relevant time period after manufacturing, such as for about 1, 3, 6, 12, 18, 24 or 36 months, when it is kept in its original packaging under the specified storage conditions.

The reconstituted solution or diluted solution of the present invention which is prepared by mixing lyophilized powder and suitable liquid vehicle remains stable for relevant hold time period, such as for about 1, 3, 6, 8, 12, 18, 24 or 36 hours when it is kept under the specified storage conditions.

The diluted solution of the invention which is prepared by mixing the reconstituted solution and suitable liquid diluent and remains stable for relevant hold time period, such as for about 1, 3, 6, 8, 12, 18, 24 or 36 hours when it is kept under the specified storage conditions.

The term “lyophilizable” means a solution which is able to go under lyophilization process using conventional methods and provides lyophilized powder or cake upon lyophilization.

As used herein, the term “micelle” means an aggregate of surfactant and/or phospholipid molecules, normally spherical in shape, wherein the aggregate have hydrophobic center and hydrophilic periphery.

As used herein, the term “drug-phospholipid micelle” means a micelle formed by drug and phospholipid molecules, wherein drug molecules are present in solubilized form in the hydrophobic core of the micelle.

As used herein, the term “docetaxel-DMPG micelle” means a micelle, comprising docetaxel and DMPG molecules, wherein docetaxel molecules are present in solubilized form in the hydrophobic core of the micelle.

As used herein, the term “cabazitaxel-DMPG micelle” means a micelle comprising cabazitaxel and DMPG molecules, wherein cabazitaxel molecules are present in solubilized form in the hydrophobic core of the micelle.

As used herein, the term “clear solution” means a solution which does not comprise any visible particulate matter, liposome or nanoparticles. The clear solution provides absorbance, when measured at 420 nm, not more than 0.1 AU (absorbance unit), for example, not more than 0.05 AU, not more than 0.04 AU or not more than 0.03 AU. The clear solution provides % transmittance, when measured at 650 nm, not less than 97%, for example, not less than 98%, not less than 99%, not less than 99.5%, not less than 99.6%, not less than 99.7% or not less than 99.8%.

As used herein, the term “powder for injection” means powder to prepare liquid composition suitable for parenteral administration. Such liquid composition may be prepared by mixing the powder with one or more of suitable liquid diluents such as water, dextrose, saline and the like.

As used herein, the term “buffer” means a chemical agent able to absorb a certain quantity of acid or base without undergoing a strong variation in the pH.

### Abbreviations:

RT=Room temperature

RH=Relative humidity

1M, 2M and 3M=1 month, 2 months and 3 months, respectively.

ICH=International Conference on Harmonisation

ND=Not detected

NA=Not analyzed

CRT=Controlled room temperature

BQL=Below quantification level/limit

° C.=Degree Celsius (unit of temperature)

μ=Micrometer (unit of length)

The suitable pharmaceutically acceptable excipients for the composition of the present invention may include one or more of the pharmaceutically acceptable solvents, phospholipids, polymers, pH adjusting agents, stabilizers, buffers, cryoprotectants, preservatives, isotonicity adjusting agents, surfactants, and anti-oxidants.

Examples of solvents may include, but not limited to, water for injection, mixture of water for injection with one or more of dehydrated alcohol, propylene glycol, polyethylene glycol, test butyl alcohol, acetonitrile, dimethyl acetamide or glycerine and the like.

Examples of phospholipids may include, but not limited to, phosphatidylcholines such as 1,2-dimyristoyl-sn-glycero-3-phosphocholine (DMPC), 1,2-dioleoyl-sn-glycero-3-phosphocholine (DOPC) and the like; phosphatidylglycerols such as 1,2-dimyristoyl-sn-glycero-3-phosphorylglycerol, sodium (DMPG Na), 1,2-dipalmitoyl-sn-glycero-3-phosphorylglycerol, sodium (DPPG Na), 1,2-distearoyl-sn-glycero-3-phosphorylglycerol, sodium (DSPG Na), 1,2-dioleoyl-sn-glycero-3-phosphorylglycerol, sodium (DOPG Na) and the like; phosphatidylserines such as 1,2-dimyristoyl-sn-glycero-3-phosphoserine, sodium (DMPS Na), 1,2-dipalmitoyl-sn-glycero-3-phosphoserine, sodium (DPPS Na), 1,2-distearoyl-sn-glycero-3-phosphoserine, sodium (DSPS Na), 1,2-dioleoyl-sn-glycero-3-phosphoserine, sodium (DOPS Na) and the like; phosphatidic acids such as 1,2-dimyristoyl-sn-glycero-3-phosphatidic acid, sodium (DMPA Na), 1,2-dipalmitoyl-sn-glycero-3-phosphatidic acid, sodium (DPPA Na), 1,2-distearoyl-sn-glycero-3-phosphatidic acid, sodium (DSPA Na), 1,2-dioleoyl-sn-glycero-3-phosphatidic acid, sodium (DOPA Na) and the like; phosphatidylethanolamines such as 1,2-dimaristoyl-sn-glycero-3-phosphoethanolamine (DMPE), 1,2-dioleoyl-sn-glycero-3-phosphoethanolamine (DOPE) and the like; methoxy conjugated phospholipids such as N-(Carbonyl-methoxypolyethyleneglycol 2000)-1,2-distearoyl-sn-glycero-3-phosphoethanolamine, sodium (mPEG₂₀₀₀-DSPE), N-(Carbonyl-methoxy polyethyleneglycol 5000)-1,2-distearoyl-sn-glycero-3-phosphoethanolamine, sodium (mPEG₅₀₀₀-DSPE), N-(Carbonyl-methoxypolyethyleneglycol 2000)-1,2-dipalmitoyl-sn-glycero-3-phosphoethanolamine, sodium (mPEG₂₀₀₀-DPPE), N-(Carbonyl-methoxypolyethyleneglycol 5000)-1,2-dipalmitoyl-sn-glycero-3-phosphoethanolamine, sodium (mPEG₅₀₀₀-DPPE) and the like, or any combination thereof. The amount of the phospholipid present in the composition may vary and depends on the nature and type of the drug as well as amount of drug to be administered, nature and type of the phospholipid, type of interaction between drug and phospholipid molecules in the aqueous phase, etc. For example, amount of Dimyristoyl phosphatidylglycerol (DMPG) may be such that the solution contains concentration of DMPG between 1 mg/mL and 50 mg/mL. For example, amount of N-(Carbonyl-methoxypolyethylene glycol 2000)-1,2-distearoyl-sn-glycero-3-phosphoethanolamine, sodium salt (mPEG₂₀₀₀-DSPE) may be such that the solution contains concentration of mPEG₂₀₀₀-DSPE between 1 mg/mL and 50 mg/mL.

Examples of pH adjusting agents may include, but not limited to, sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, sodium carbonate, sodium bicarbonate, triethanolamine, or any combination thereof.

Examples of stabilizers may include, but not limited to, polyvinylpyrrolidone, polyvinyl alcohol, polyethylene glycol, diethanolamine, ferric chloride, inositol, sodium gluconate, creatinine, glycerine, niacinamide, sodium saccharin, sodium caprylate, arginine, methionine, cysteine and the like, or any combination thereof.

Examples of buffers may include, but not limited to, acetate (e.g. sodium acetate etc.), citrate (e.g. citric acid/sodium citrate etc.), phosphate (e.g. monobasic sodium phosphate, dibasic sodium phosphate etc.), carbonate, or any combination thereof.

Examples of cryoprotectants may include, but not limited to, sucrose, lactose, mannitol, polyethylene glycol, polyvinylpyrrolidone, or any combination thereof.

Examples of preservatives may include, but not limited to, chlorobutanol, benzalkonium chloride, methyl paraben, propyl paraben, benzoic acid, sodium benzoate, sorbic acid, benzethonium chloride, cetyl pyridinium chloride, benzyl bromide, benzyl alcohol phenylmercury nitrate, phenylmercury acetate, thiomersal, merthiolate, chlorhexidine, phenylethyl alcohol, quaternary ammonium chloride, sodium benzoate, sodium propionate, or any combination thereof.

Examples of isotonicity adjusting agents may include, but not limited to, sodium chloride, potassium chloride, calcium chloride, magnesium chloride, glucose, sucrose, dextrose, mannitol, glycerol, or any combination thereof.

Examples of suitable pharmaceutically acceptable surfactants may include, but not limited to, amphoteric, non-ionic, cationic or anionic molecules. Suitable surfactants may include, but not limited to, polysorbates (e.g. tween 80 etc.), poloxamer (poloxamer 188), sodium lauryl sulfate, lauryl dimethyl amine oxide, docusate sodium, cetyl trimethyl ammonium bromide (CTAB), polyethoxylated alcohols, polyoxyethylene sorbitan, octoxynol, polyoxyl lauryl ether, polyoxyethylene vegetable-based fatty ethers derived from lauryl, cetyl, stearyl and oleyl alcohols, bile salts (such as sodium deoxycholate and sodium cholate), polyoxyl castor oil, nonylphenol ethoxylate, lecithin, polyoxyethylene surfactants, polyethylene glycol esters, glycol, esters of fatty acids, monoalkanolamine condensates, polyoxyethylene fatty acid amides, quaternary ammonium salts, polyoxyethylene alkyl and alicyclic amines, polyoxyethylene, sorbitan monolaurate and stearate, Cremophor® polyethoxylated castor oil), Solutol® (ethylene oxide/12-hydroxy stearic acid), tyloxapol, or any combination thereof.

Examples of suitable pharmaceutically acceptable anti-oxidants may include, but not limited to, butylated hydroxytoluene (BHT), butylated hydroxyanisole (BHA), and propyl gallate (PG), monothioglycerol, ascorbic acid, citric acid, tartaric acid, sodium ascorbate, erythorbic acid, potassium metabisulfite, sodium metabisulfite, propionic acid, sodium formaldehyde sulphoxylate, reduced glutathione, thiourea, cysteine, n-acetylcysteine, methionine, sodium sulfite, alkyl gallate, including propyl gallate, vitamin E, or other tocopherol analogs, including tocopherol acetate or TPGS, or any combination thereof.

The sterile injectable compositions of the present invention may have improved physical and/or chemical stability. The stable lyophilized powder composition of the present invention may retain at least 90% potency of the drug after storage for more than 6 months, for example, for 12 months, 18 months, 24 months or 36 months, at 2-8° C.

The stable lyophilized powder composition of the present invention may retain at least 90% potency of the drug after storage for more than 6 months, for example, for 12 months, 18 months, 24 months or 36 months, at 25° C.±2° C. and 60% RH (relative humidity). The stable lyophilized powder composition of the present invention may retain at least 90% potency of the drug after storage for more than 6 months, for example, for 12 months, 18 months, 24 months or 36 months, at 40° C.±2° C. and 75% RH. The stable, clear, aqueous solution of the present invention may retain at least 90% potency of the drug after storage for 6 hours or more, for example, storage for 8 hours, 12 hours, 18 hours, 24 hours, 36 hours or 48 hours, at 2-8° C.

The stable, clear, aqueous solution of the present invention may retain at least 90% potency of the drug after storage for 6 hours or more, for example, storage for 8 hours, 12 hours, 18 hours, 24 hours, 36 hours or 24 hours, at 25° C.

In one embodiment, there is provided a stable lyophilized powder comprising docetaxel or cabazitaxel, which remains stable and compliant as per ICH guideline for impurities for respective product, for more than 2 months, for example, for 3 months, for 6 months, for 12 months, for 24 months or for 36 months, when stored at 25° C.±2° C. temperature and 60% RH or at 2-8° C. temperature.

In another embodiment of the invention, there is provided a stable lyophilized powder comprising docetaxel which does not contain more than 1% of 10-docetaxel baccatin, more than 1% of 2-debenzoxyl 2-pentenoyl docetaxel, more than 1% of 6-oxodocetaxel, more than 1% of 4-epidocetaxel, more than 1% of 4-epi-6-docetaxel, more than 1% of the single maximum unknown impurity and/or more than 2% (for example, 1% or 0.5%) of the total impurities when stored at 2-8° C. temperature or at 25° C. temperature and 60% RH or at 30° C. temperature and 65% RH or at 40° C. temperature and 75% RH, for more than 2 months, for example, for 3 months, for 6 months, for 12 months, for 18 months, for 24 months or for 36 months.

In another embodiment of the invention, there is provided a stable lyophilized powder comprising cabazitaxel which does not contain more than 1% of impurity A, more than 1% of impurity B, more than 1% of the single maximum unknown impurity and/or more than 2% (for example, 1% or 0.5%) of the total impurities when stored at 2-8° C. temperature or at 25° C. temperature and 60% RH or at 30° C. temperature and 65% RH or at 40° C. temperature and 75% RH, for more than 2 months, for example, for 3 months, for 6 months, for 12 months, for 18 months, for 24 months or for 36 months. For cabazitaxel, impurity A is 4α-Acetoxy-2α-benzoyloxy-5β, 20-epoxy-β, 13α-dihydroxy-7β, 10β-dimethoxy-9oxo-11-taxene and impurity B is 4α-Acetoxy-2α-benzoyloxy-5β, 20-epoxy-1β, 13α-dihydroxy-7β, 10β-dimethoxy-9oxo-11-taxene-13α-yl (2R, 4S, 5R)-3-tert-buloxycarbonyl-2-(4-methoxyphenyl)-4-phenyl-1,3-Oxazolidine-5-carboxylate.

In one embodiment, there is provided a stable, clear, aqueous solution comprising docetaxel or cabazitaxel, which remains stable and compliant as per ICH guideline for impurities for respective product, for more than 4 hours, for example, for 8 hours, for 12 hours, for 18 hours, 24 hours or for 36 hours, when stored at 2-8° C. temperature or at 25° C.±2° C. temperature and 60% RH.

In another embodiment of the invention, there is provided a stable, clear, aqueous solution comprising docetaxel which does not contain more than 1% of 10-docetaxel baccatin, more than 1% of 2-debenzoxyl 2-pentenoyl docetaxel, more than 1% of 6-oxodocetaxel, more than 1% of 4-epidocetaxel, more than 1% of 4-epi-6-docetaxel, more than 1% of the single maximum unknown impurity and/or more than 2% (for example, 1% or 0.5%) of the total impurities when stored at 2-8° C. temperature or at 25° C. (room temperature) for more than 4 hours, for example, for 6 hours, for 8 hours, for 10 hours, for 12 hours, for 18 hours, for 24 hours or for 48 hours. The stable, clear, aqueous solution comprising docetaxel does not form precipitate and remains physically stable up to 6 hours, 8 hours, 10 hours, 12 hours, 18 hours, 24 hours, 36 hours or 48 hours when stored at 2-8° C. temperature or at 25° C.±2° C. temperature and 60% RH.

In another embodiment of the invention, there is provided a stable, clear, aqueous solution comprising cabazitaxel which does not contain more than 1% of impurity A, more than 1% of impurity B, more than 1% of the single maximum unknown impurity and/or more than 2% (for example, 1% or 0.5%) of the total impurities when stored at 2-8° C. temperature or at 25° C. (room temperature) for more than 4 hours, for example, for 6 hours, for 8 hours, for 10 hours, for 12 hours, for 18 hours, for 24 hours or for 48 hours. The stable, clear, aqueous solution comprising cabazitaxel does not form precipitate and remains physically stable up to 6 hours, 8 hours, 10 hours, 12 hours, 18 hours, 24 hours, 36 hours or 48 hours when stored at 2-8° C. temperature or at 25° C.±2° C. temperature and 60% RH.

In one embodiment, the present invention provides a process for the preparation of the lyophilized powder comprising one or more drugs and one or more phospholipids. The process includes the steps of (a) preparing aqueous solution comprising one or more phospholipids; (b) preparing drug non-aqueous solution comprising one or more drugs; (c) mixing the solution of step (a) and step (b); and (d) lyophilizing so obtained solution mixture. So obtained lyophilized powder may be reconstituted using an appropriate quantity of water for injection (or other suitable vehicle) to provide a stable, clear, aqueous solution for intravenous administration. The reconstituted solution may be further diluted using an appropriate solution such as 5% dextrose, saline etc., for parenteral administration.

In another embodiment, the present invention provides a process for the preparation of the lyophilized powder comprising docetaxel and DMPG. The process includes the steps of (a) preparing aqueous solution comprising DMPG Na; (b) preparing non-aqueous solution comprising docetaxel; (c) mixing the solution of step (a) and step (b); and (d) lyophilizing so obtained solution mixture to obtain lyophilized powder.

Additionally, PVP or other stabilizer may be added at step (a), (b) or (c). Additionally, mPEG₂₀₀₀-DSPE may be added at step (a). So obtained powder at step (d) may be reconstituted using an appropriate quantity of water for injection to provide a clear solution comprising docetaxel and DMPG which may be present in the form of docetaxel-DMPG micelles. The reconstituted solution may be further diluted using an appropriate solution such as 5% dextrose, saline etc., for intravenous administration.

In another embodiment, the present invention provides a process for the preparation of the lyophilized powder comprising cabazitaxel and DMPG. The process includes the steps of (a) preparing aqueous solution comprising DMPG Na; (b) preparing non-aqueous solution comprising cabazitaxel; (c) mixing the solution of step (a) and step (b); and (d) lyophilizing so obtained solution mixture to obtain lyophilized powder.

Additionally, PVP or other stabilizer may be added at step (a), (b) or (c). Additionally, mPEG₂₀₀₀-DSPE may be added at step (a). So obtained powder may be reconstituted using an appropriate quantity of water for injection to provide a clear solution comprising cabazitaxel and DMPG which may be present in the form of cabazitaxel-DMPG micelles. The reconstituted solution may be further diluted using an appropriate solution such as 5% dextrose, saline etc., for intravenous administration. The following examples are for the purpose of illustration of the invention only and are not intended in any way to limit the scope of the present invention.

### EXAMPLE 1

TABLE 1

| Ingredients | Quantity/batch | |
|---|---|---|
| Docetaxel | 250 | mg |
| 1,2-dimyristoyl-sn-glycero-3-phosphorylglycerol sodium | 1500 | mg |
| salt (DMPG Na) | | |
| N-(Carbonyl-methoxypolyethyleneglycol 2000)-1,2- | 500 | mg |
| distearoyl-sn-glycero-3-phosphoethanolamine, sodium | | |
| salt (mPEG₂₀₀₀-DSPE) | | |
| Polyvinylpyrrolidone K-12 | 500 | mg |
| Dehydrated alcohol | 3 | mL |
| Sucrose | 10 | gram |

| | |
|---|---|
| Hydrochloric acid | q.s. |
| Sodium Hydroxide | q.s. |
| Water for Injection (WFI) | q.s. to 100 mL |

### Process

### Manufacturing Steps:

(a) Aqueous Solution

Accurately weighed 1500 mg of DMPG Na was added to an appropriate quantity of water for injection at 50° C. under stirring and continued to stir till it became clear solution.

To the obtained clear solution, accurately weighed 500 mg of mPEG₂₀₀₀-DSPE and 10 gram of the sucrose were added and stirred until dissolved.

The volume of so obtained aqueous solution was made up with water for injection up to 95 mL and filtered through 0.22 μ filter.
(b) Non-Aqueous Solution

Accurately weighed 250 mg of docetaxel and 500 mg of the polyvinylpyrrolidone were added to 2 mL of dehydrated alcohol under stirring and continued to stir till it became clear solution.
(c) Homogeneous Solution

Filtered 95 mL of the aqueous solution obtained in step (a) was added to the non-aqueous solution obtained in step (b) under stirring and continued to stir till it became clear solution. Volume was adjusted up to 100 mL using water for injection.

Then after pH was checked and adjusted using appropriate quantities of hydrochloric acid and sodium hydroxide to have pH of aqueous solution about 8.

Thus obtained homogeneous solution was filtered through 0.22 μ filter to obtain ready for lyophilization solution.
(d) Lyophilization

Thus obtained ready for lyophilization solution was filled into 20 mL glass vials, 8 mL each, so that each vial contains 20 mg of docetaxel. So obtained glass vials were lyophilized to obtain lyophilized powder composition. In the similar way, lyophilized powder composition containing 40 mg of docetaxel in a vial may be obtained by lyophilization of a glass vial filled with 16 mL of the homogeneous solution.
(e) Reconstitution

Thus obtained lyophilized powder in above step (d) was reconstituted using water for injection to provide reconstituted solution having 2.5 mg/mL docetaxel.
(f) Dilution

Thus obtained reconstituted solution in above step (e) was further diluted using 5% dextrose to provide diluted solution having 0.33 mg/mL docetaxel.

The ready for lyophilization solution obtained in above mentioned step (c), lyophilized powder obtained in above mentioned step (d), reconstituted solution obtained in above mentioned step (e) and diluted solution obtained in above mentioned step (f) were tested for their stability and other parameters and results were reported in below Table 2 through Table 7.

TABLE 2
Physical stability of ready for lyophilization
solution, obtained at step (c) of Example 1

| | | CRT (controlled |
|---|---|---|
| Concentration | 2-8° C. | room temperature) |
| 2.5 mg/mL docetaxel | Clear at 48 hour | Clear solution |
| | | at 24 hour |

TABLE 3
Chemical stability of ready for lyophilization solution, obtained at step (c) of Example 1

| | Storage conditions | | | | |
|---|---|---|---|---|---|
| | | 2-8° C. | | CRT | |
| Tests | Initial | 6 h | 24 h | 6 h | 24 h |
| Docetaxel assay (%) | 102.9 | 102.8 | 102.7 | 102.2 | 102.3 |
| 10-Docetaxel baccatin (%) | ND | ND | ND | ND | ND |
| 2-debenzoxyl 2-pentenoyl docetaxel (%) | BQL | BQL | BQL | BQL | BQL |
| Crotonaldehyde analog | ND | ND | ND | ND | ND |
| 6-Oxodocetaxel (%) | 0.1 | 0.1 | 0.09 | 0.08 | 0.06 |
| 4-Epidocetaxel (%) | 0.11 | 0.12 | 0.11 | 0.13 | 0.22 |
| 4-Epi-6-Docetaxel (%) | ND | ND | ND | ND | ND |
| Individual maximum unknown impurity (%) | BQL | BQL | BQL | BQL | BQL |
| Total impurities (%) | 0.25 | 0.22 | 0.22 | 0.21 | 0.28 |

TABLE 4
Description and chemical stability of lyophilized powder, obtained at step (d) of Example 1

| | | storage conditions | | |
|---|---|---|---|---|
| | | 2-8° C. | 25° C./60% RH | 30° C./65% RH |
| Parameters | Initial | 3 M | 3 M | 3 M |
| Description | White to off- | White to off- | White to off- | White to off- |
| | white Colored | white colored | white colored | white colored |
| | lyophilized cake | lyophilized cake | lyophilized cake | lyophilized cake |
| Docetaxel assay (%) | 100.4 | 101.5 | 98.3 | 99.1 |
| 10-Docetaxel baccatin (%) | ND | ND | ND | ND |
| 2-debenzoxyl 2-pentenoyl | NA | BQL | BQL | BQL |
| docetaxel (%) | | | | |
| 6-Oxodocetaxel (%) | 0.06 | 0.04 | 0.03 | BQL |
| 4-Epidocetaxel (%) | 0.09 | 0.14 | 0.79 | 1 |
| 4-Epi-6-Docetaxel (%) | NA | BQL | 0.05 | 0.06 |
| Individual maximum | NA | BQL | BQL | BQL |
| unknown impurity (%) | | | | |
| Total impurities (%) | 0.15 | 0.18 | 0.87 | 1.1 |
| Water content (%) | 0.99 | NA | NA | NA |

TABLE 5
Physical stability of reconstituted solution,
obtained at step (e) of Example 1

| Reconstitution | | | |
|---|---|---|---|
| vehicle | Concentration | 2-8° C. | CRT |
| Water for injection | 2.5 mg/mL | Clear solution | Clear solution |
| | docetaxel | at 8 hour | at 8 hour |

TABLE 6
Appearance, Absorbance and % Transmittance of reconstituted
solution, obtained at step (e) of Example 1

| Appearance | Clear colorless solution |
|---|---|
| Absorbance at 420 nm | 0.025 |
| % Transmittance at 650 nm | 99.89 |

TABLE 7
Physical stability of diluted solution,
obtained at step (f) of Example 1

| Dilution | | | |
|---|---|---|---|
| vehicle | Concentration | 2-8° C. | CRT |
| 5% dextrose | 0.33 mg/mL | Clear solution | Clear solution |
| | docetaxel | at 48 hour | at 24 hour |

### EXAMPLE 2

TABLE 8

| Ingredients | Quantity/batch | |
|---|---|---|
| Cabazitaxel | 250 | mg |
| 1,2-dimyristoyl-sn-glycero-3-phosphorylglycerol | 1500 | mg |
| sodium salt (DMPG) | | |
| N-(Carbonyl-methoxypolyethyleneglycol 2000)- | 500 | mg |
| 1,2-distearoyl-sn-glycero-3-phosphoethanolamine, | | |
| sodium salt (mPEG₂₀₀₀-DSPE) | | |
| Polyvinylpyrrolidone K-12 | 500 | mg |
| Dehydrated alcohol | 3 | mL |
| Sucrose | 10 | gram |

| | |
|---|---|
| Hydrochloric acid | q.s. |
| Sodium hydroxide | q.s. |
| Water for Injection (WFI) | q.s. to 100 mL |

### Process

### Manufacturing Steps

(a) Aqueous Solution

Accurately weighed 1500 mg of DMPG Na was added to an appropriate quantity of water for injection at 50° C. under stirring and continued to stir till it became clear solution.

To the obtained clear solution, accurately weighed 500 mg of mPEG₂₀₀₀-DSPE and 10 gram of sucrose were added and stirred until dissolved.

Then after, pH was adjusted using appropriate quantities of hydrochloric acid and sodium hydroxide to have pH of aqueous solution about 6.

The volume of so obtained aqueous solution was made up with water for injection up to 95 mL and filtered through 0.22 μ filter.
(b) Non-Aqueous Solution

Accurately weighed 250 mg of cabazitaxel and 500 mg of polyvinylpyrrolidone were added to 3 mL of dehydrated alcohol under stirring and continued to stir till it became clear solution.
(c) Homogeneous Solution

Filtered 95 mL of the aqueous solution obtained in step (a) was added to the non-aqueous solution obtained in step (b) under stirring and continue to stir till it became clear homogeneous solution. Volume was adjusted up to 100 mL using water for injection.

Thus obtained homogeneous solution was filtered through 0.22 μ filter to obtain ready for lyophilization solution.
(d) Lyophilization

Thus obtained ready for lyophilization solution was filled into 50 mL glass vials, 24 mL each, so that each vial contains 60 mg of cabazitaxel. So obtained glass vials were lyophilized to obtain lyophilized powder composition.
(e) Reconstitution

Thus obtained lyophilized powder in above step (d) was reconstituted using water for injection to provide reconstituted solution having 2.5 mg/mL cabazitaxel.
(f) Dilution

Diluted solution 1—0.1 mg/mL

Thus obtained reconstituted solution in above step (e) was further diluted using 5% dextrose to provide diluted solution having 0.1 mg/mL cabazitaxel.

Diluted solution 2—0.26 mg/mL

Thus obtained reconstituted solution is above step (e) was further diluted using 5% dextrose to provide diluted solution having 0.26 mg/mL cabazitaxel.

The ready for lyophilization solution obtained in above mentioned step (c), lyophilized powder obtained in above mentioned step (d), reconstituted solution obtained in above mentioned step (e) and diluted solutions obtained in above mentioned step (f) were tested for their stability and other parameters and results were reported in below Table 9 through Table 16.

TABLE 9
Physical stability of ready for lyophilization
solution, obtained at step (c) of Example 2

| Concentration | 2-8° C. | CRT |
|---|---|---|
| 2.5 mg/mL cabazitaxel | Clear at 48 hour | Clear solution |
| | | at 24 hour |

TABLE 10
Chemical stability of ready for lyophilization
solution, obtained at step (c) of Example 2

| | Storage conditions | | | |
|---|---|---|---|---|
| | | 2-8° C. | | CRT |
| Tests | Initial | 8 h | 24 h | 8 h |
| Cabazitaxel assay (%) | 101.1 | 102 | 101.8 | 102.3 |
| Impurity A (%) | BQL | BQL | BQL | BQL |
| Impurity B (%) | 0.06 | 0.07 | 0.07 | 0.07 |
| Single maximum | BQL | BQL | BQL | BQL |
| unknown impurity (%) | | | | |
| Total Impurities (%) | 0.06 | 0.07 | 0.07 | 0.07 |

TABLE 11
Description and chemical stability of lyophilized powder, obtained at
step (d) of Example 2

| | Storage condition | | | | | | |
|---|---|---|---|---|---|---|---|
| Parameter | | 25° C. and 60% RH | | | 40° C. and 75% RH | | |
| measured | Initial | 1 M | 2 M | 3 M | 1 M | 2 M | 3 M |
| Description | White to off-white colored lyophilized cake | | | | | | |
| Cabazitaxel | 100.10 | 99.80 | 100.40 | 100.20 | 100.90 | 101.00 | 100.90 |
| assay (%) | | | | | | | |
| Impurity A (%) | ND | BQL | BQL | BQL | BQL | BQL | BQL |
| Impurity B (%) | 0.05 | 0.06 | 0.06 | 0.07 | 0.06 | 0.07 | 0.1 |
| Single | BQL | 0.06 | 0.06 | 0.06 | 0.1 | 0.17 | 0.16 |
| maximum | | | | | | | |
| unknown | | | | | | | |
| impurity (%) | | | | | | | |
| Total | 0.05 | 0.12 | 0.12 | 0.13 | 0.23 | 0.44 | 0.61 |
| Impurities (%) | | | | | | | |
| pH | 5.75 | — | — | 5.78 | — | — | — |
| Water content | 0.76 | — | — | 0.75 | — | — | — |
| (%) | | | | | | | |

TABLE 12
Physical stability of reconstituted solution,
obtained at step (e) of Example 2

| Reconstitution | | | |
|---|---|---|---|
| vehicle | Concentration | 2-8° C. | 25° C. |
| Water for injection | 2.5 mg/mL | Clear solution | Clear solution |
| | cabazitaxel | at 48 hour | at 24 hour |

TABLE 13
Chemical stability of reconstituted solution, obtained at step (e) of
Example 2

| | | | | | | | Total | |
|---|---|---|---|---|---|---|---|---|
| | Cabazitaxel | | Impurity A | | Impurity B | | Impurities | |
| Time | Assay (%) | | (%) | | (%) | | (%) | |
| point | 2-8° C. | 25° C. | 2-8° C. | 25° C. | 2-8° C. | 25° C. | 2-8° C. | 25° C. |
| Initial | 96.5 | 96.5 | BQL | BQL | 0.06 | 0.06 | 0.06 | 0.06 |
| 8 hours | 97.3 | 98.5 | BQL | BQL | 0.06 | 0.06 | 0.06 | 0.06 |
| 24 hours | 97.8 | 97.5 | BQL | BQL | 0.06 | 0.06 | 0.06 | 0.06 |

TABLE 14
Physical stability of diluted solution 1 (0.1
mg/mL), obtained at step (f) of Example 2

| Dilution | | | |
|---|---|---|---|
| vehicle | Concentration | 2-8° C. | CRT |
| 5% dextrose | 0.1 mg/mL | Clear solution | Clear solution |
| | cabazitaxel | at 48 hour | at 24 hour |

TABLE 15
Chemical stability of diluted solution 1 (0.1 mg/mL), obtained at step (f)
of Example 2

| | Cabazitaxel | | | | | | | |
|---|---|---|---|---|---|---|---|---|
| | assay (%) | | | | | | | |
| | with respect | | | | | | Total | |
| | to the initial | | Impurity A | | Impurity B | | Impurities | |
| Time | time point | | (%) | | (%) | | (%) | |
| point | 2-8° C. | 25° C. | 2-8° C. | 25° C. | 2-8° C. | 25° C. | 2-8° C. | 25° C. |
| Initial | 100.00 | 100.00 | BQL | BQL | 0.05 | 0.05 | 0.05 | 0.05 |
| 8 | 100.11 | 100.56 | BQL | BQL | 0.05 | 0.06 | 0.05 | 0.06 |
| hours | | | | | | | | |
| 24 | 100.34 | 99.66 | BQL | BQL | 0.05 | 0.05 | 0.05 | 0.05 |
| hours | | | | | | | | |

TABLE 16
Physical stability of diluted solution 2 (0.26
mg/mL), obtained at step (f) of Example 2
Physical stability

| 2-8° C. | 25° C. |
|---|---|
| Clear solution at 48 hour | Clear solution at 24 hour |

TABLE 17
Chemical stability of diluted solution 2 (0.26 mg/mL), obtained at step (f)
of Example 2

| | Cabazitaxel | | | | | | | |
|---|---|---|---|---|---|---|---|---|
| | assay (%) with | | | | | | Total | |
| | respect to the | | Impurity A | | Impurity B | | Impurities | |
| Time | initial time point | | (%) | | (%) | | (%) | |
| point | 2-8° C. | 25° C. | 2-8° C. | 25° C. | 2-8° C. | 25° C. | 2-8° C. | 25° C. |
| Initial | 100 | 100 | BQL | BQL | 0.05 | 0.05 | 0.05 | 0.05 |
| 8 | 101.83 | 103.09 | BQL | BQL | 0.05 | 0.05 | 0.05 | 0.05 |
| hours | | | | | | | | |
| 24 | 102.17 | 102.52 | BQL | BQL | 0.05 | 0.05 | 0.05 | 0.05 |
| hours | | | | | | | | |

While the present invention has been described in terms of its specific embodiments, certain modifications and equivalents will be apparent to those skilled in the art and are intended to be included within the scope of the present invention.

1-40. (canceled)

41. A sterile injectable composition comprising one or more drugs, 1,2-dimyristoyl-sn-glycero-3-phosphorylglycerol (DMPG), N-(carbonyl-methoxypolyethyleneglycol)-1,2-distearoyl-sn-glycero-3-phosphoethanolamine (mPEG₂₀₀₀-DSPE) and one or more stabilizers.

42. The sterile injectable composition according to claim 41, wherein the stabilizer is polyvinylpyrrolidone (PVP).

43. The sterile injectable composition according to claim 41, wherein the drug is docetaxel, paclitaxel, cabazitaxel, temsirolimus, carmustine, bendamustine, melphalan, busulphan, carfilzomib, bortezomib, teniposide, ixabepilone, sirolimus, tacrolimus or everolimus.

44. The sterile injectable composition according to claim 41, wherein the composition is a clear aqueous solution.

45. The sterile injectable composition according to claim 44, wherein the solution is lyophilizable.

46. The sterile injectable composition according to claim 44, wherein the solution further comprises one or more pH adjusting agents.

47. The sterile injectable composition according to claim 41, wherein the composition is a lyophilized powder for injection.

48. The sterile injectable composition according to claim 47, wherein the lyophilized powder provides a clear solution when reconstituted with one or more aqueous vehicles.

49. The sterile injectable composition according to claim 48, wherein the solution has % transmittance not less than 99%, when measured at 650 nm.

50. The sterile injectable composition according to claim 48, wherein the solution has absorbance not more than 0.1 AU (absorbance unit), when measured at 420 nm.

51. A sterile injectable composition comprising docetaxel and 1,2-dimyristoyl-sn-glycero-3-phosphorylglycerol (DMPG), wherein the composition does not comprise cholesterol or its derivatives.

52. The sterile injectable composition according to claim 51 further comprises N-(carbonyl-methoxypolyethyleneglycol)-1,2-distearoyl-sn-glycero-3-phosphoethanolamine (mPEG₂₀₀₀-DSPE) and one or more stabilizers.

53. The sterile injectable composition according to claim 51, wherein the composition is a clear aqueous solution or a lyophilized powder for injection.

54. The sterile injectable composition according to claim 53, wherein the solution does not contain more than 1% of total impurities after storage for 3 months at 25° C. temperature and 60% RH.

55. The sterile injectable composition according to claim 53, wherein the lyophilized powder does not contain more than 1% of total impurities after storage for 3 months at 25° C. temperature and 60% RH.

56. A sterile injectable composition comprising cabazitaxel and 1,2-dimyristoyl-sn-glycero-3-phosphorylglycerol (DMPG).

57. The sterile injectable composition according to claim 56 further comprises N-(carbonyl-methoxypolyethyleneglycol)-1,2-distearoyl-sn-glycero-3-phosphoethanolamine (mPEG₂₀₀₀-DSPE) and one or more stabilizers.

58. The sterile injectable composition according to claim 56, wherein the composition is a clear aqueous solution or a lyophilized powder for injection.

59. The sterile injectable composition according to claim 58, wherein the solution does not contain more than 1 % of total impurities after storage for 3 months at 25° C. temperature and 60% RH.

60. The sterile injectable composition according to claim 58, wherein the lyophilized powder does not contain more than 1% of total impurities after storage for 3 months at 25° C. temperature and 60% RH.
